(19) United States
(12) Patent Application Publication
(10) Pub. No.: US 2025/0292537 A1
(43) Pub. Date: Sep. 18, 2025

(54) MEDICAL IMAGE AND TEXT PROCESSING METHOD AND APPARATUS

(71) Applicant: CANON MEDICAL SYSTEMS CORPORATION, Otawara-shi (JP)

(72) Inventors: Alison O'NEIL, Edinburgh (GB); Murray CUTFORTH, Edinburgh (GB); Antanas KASCENAS, Edinburgh (GB); Patrick SCHREMPF, Edinburgh (GB); Hannah WATSON, Edinburgh (GB)

(73) Assignee: CANON MEDICAL SYSTEMS CORPORATION, Otawara-shi (JP)

(21) Appl. No.: 18/603,404

(22) Filed: Mar. 13, 2024

(51) Int. Cl. G06V 10/74; G06F 40/279; G06T 7/00; G06V 10/77; G06V 10/774; G06V 10/776; G06V 10/80; G06V 10/82; G16H 50/50

(52) U.S. Cl. CPC G06V 10/761; G06F 40/279; G06T 7/0012; G06V 10/7715; G06V 10/774; G06V 10/776; G06V 10/806; G06V 10/82; G16H 50/50; G06T 2207/20081; G06T 2207/20084; G06T 2207/30004; G06V 2201/03

(57) ABSTRACT

An apparatus comprising processing circuitry configured to: obtain image data representing one or more medical images of a region of interest and processing said image data to identify one or more abnormal portions of the image by applying a first pre-determined model to the obtained image data; obtain medical text data corresponding to the one or more medical images of the region of interest and process said medical text data to identify one or more entities and their associated attributes by applying at least one further pre-determined model to the obtained medical text data; perform a matching process between the one or more identified abnormal portions and the one or more identified entities to obtain matched data comprising groupings of at least one identified abnormal portions and at least one entity, wherein the matching process is based on at least one or more properties of the identified abnormal portions of the image data and at least one or more of the attributes associated with the identified entities.

### FIELD

Embodiments described herein relate generally to a method and apparatus for processing text data and image data.

### BACKGROUND

Machine learning algorithms such as computer aided detection (CAD) algorithms may require large annotated or labelled training datasets. Recently available data platforms, provide access to large datasets. However, annotating such datasets for use as training data may be time-consuming and expensive, due to the requirement for specialist tools and medical experts. Platforms are available that provide access to large datasets. However, the segmentation annotations (at a pixel or voxel level) that are required to train CAD algorithms may be time-consuming and expensive to obtain, due to the requirement for specialist tools and medical experts.

### BRIEF DESCRIPTION

Embodiments are now described, by way of non-limiting example, and are illustrated in the following figures, in which:

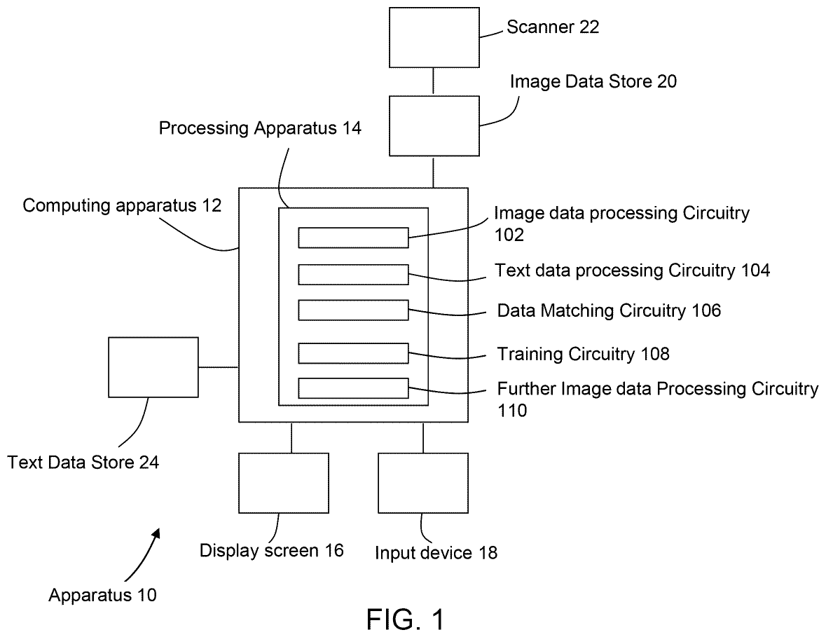

FIG. 1 is a schematic illustration of an apparatus in accordance with an embodiment;

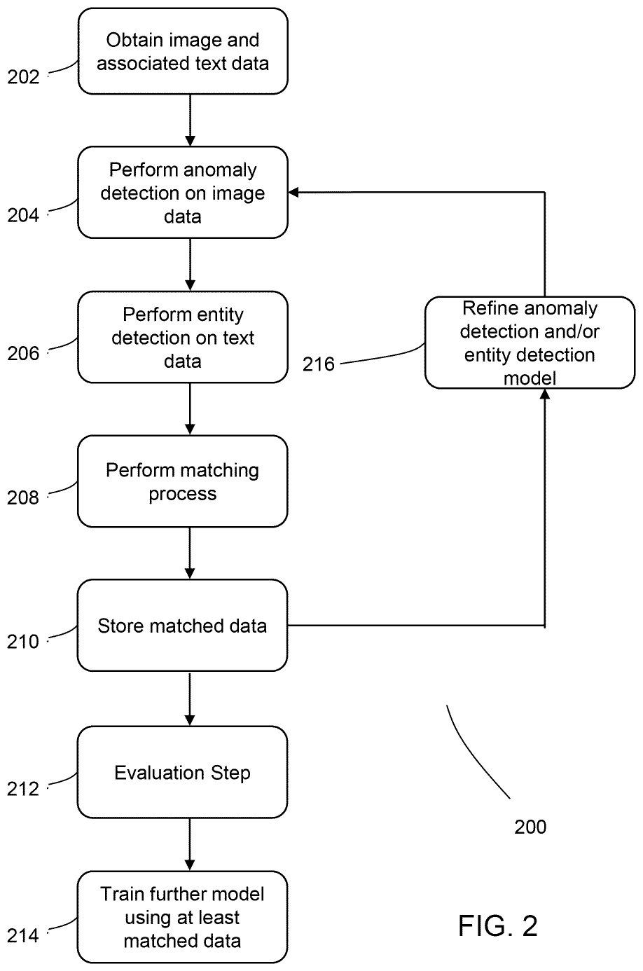

FIG. 2 is a flow chart illustrating in overview an image and text processing process in accordance with an embodiment;

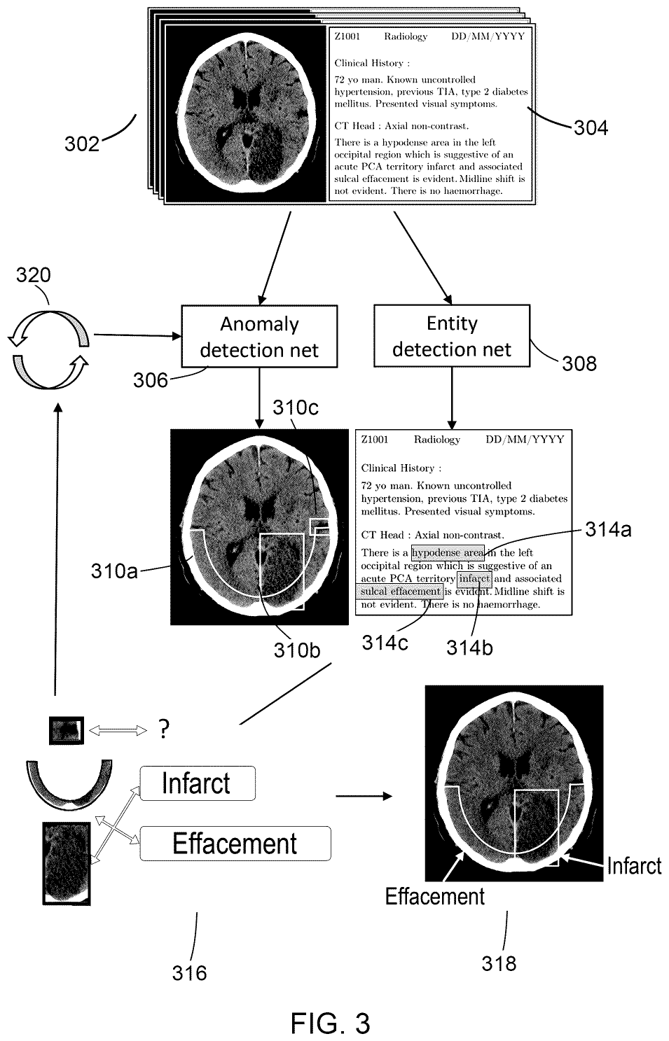

FIG. 3 schematically depicts a method, in accordance with an embodiment;

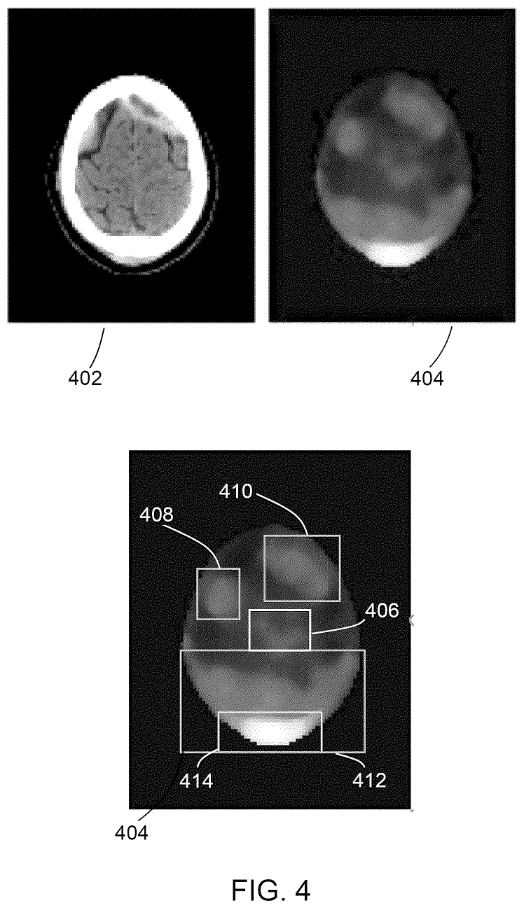

FIG. 4 depicts an abnormal portion detection process;

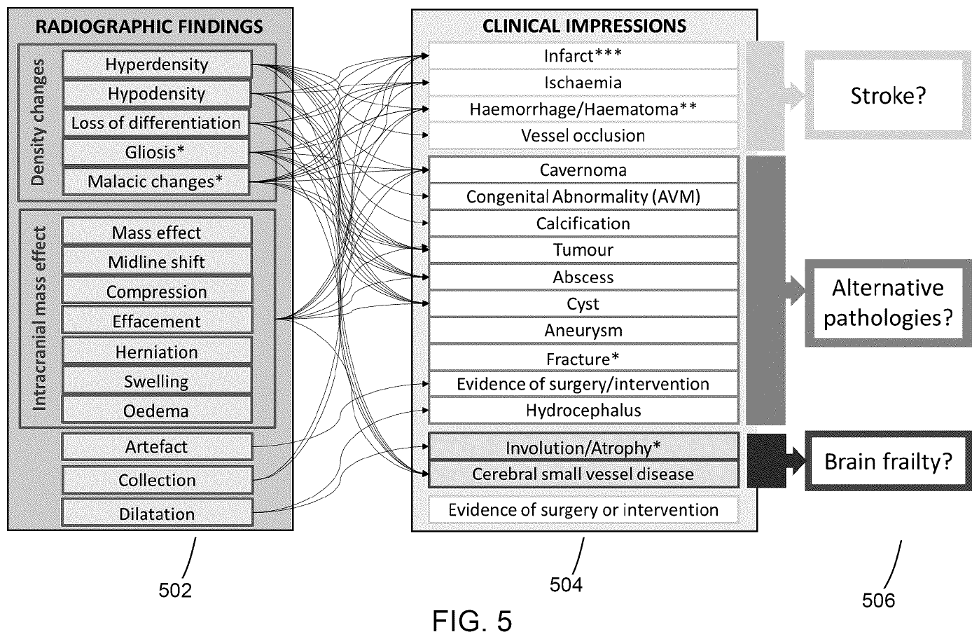

FIG. 5 depicts entities, in particular, findings and impressions, and

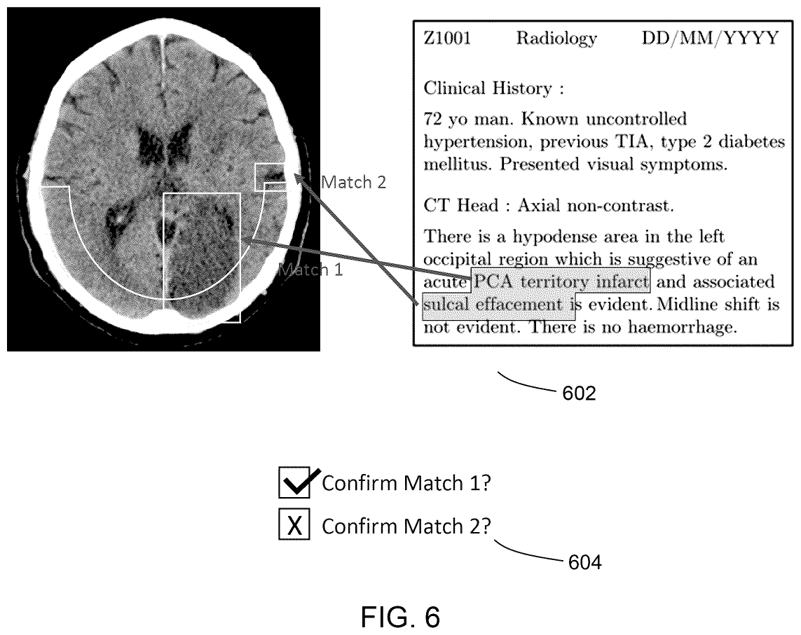

FIG. 6 is an example user interface for evaluating the output of a method, in accordance with an embodiment.

### DETAILED DESCRIPTION

Certain embodiments provide an apparatus comprising processing circuitry configured to: obtain image data representing one or more medical images of a region of interest and processing said image data to identify one or more abnormal portions of the image by applying a first pre-determined model to the obtained image data; obtain medical text data corresponding to the one or more medical images of the region of interest and process said medical text data to identify one or more entities and their associated attributes by applying at least one further pre-determined model to the obtained medical text data; perform a matching process between the one or more identified abnormal portions and the one or more identified entities to obtain matched data comprising groupings of at least one identified abnormal portions and at least one entity, wherein the matching process is based on at least one or more properties of the identified abnormal portions of the image data and at least one or more of the attributes associated with the identified entities.

Certain embodiments provide a method comprising: obtaining image data representing one or more medical images of a region of interest and processing said image data to identify one or more abnormal portions of the image by applying a first pre-determined model to the obtained image data; obtaining medical text data corresponding to the one or more medical images of the region of interest and process said medical text data to identify one or more entities and their associated attributes by applying at least one further pre-determined model to the obtained medical text data; performing a matching process between the one or more identified abnormal portions and the one or more identified entities to obtain matched data comprising groupings of at least one identified abnormal portions and at least one entity, wherein the matching process is based on at least one or more properties of the identified abnormal portions of the image data and at least one or more of the attributes associated with the identified entities.

Certain embodiments provide a non-transitory memory storing computer-readable instructions that are executable by a processor to perform the method comprising: obtaining image data representing one or more medical images of a region of interest and processing said image data to identify one or more abnormal portions of the image by applying a first pre-determined model to the obtained image data; obtaining medical text data corresponding to the one or more medical images of the region of interest and process said medical text data to identify one or more entities and their associated attributes by applying at least one further pre-determined model to the obtained medical text data; performing a matching process between the one or more identified abnormal portions and the one or more identified entities to obtain matched data comprising groupings of at least one identified abnormal portions and at least one entity, wherein the matching process is based on at least one or more properties of the identified abnormal portions of the image data and at least one or more of the attributes associated with the identified entities.

The following embodiments relate to a method of obtaining or generating matched data for the purpose of further training a machine learning model, such as a computer assisted diagnosis model. In some embodiments, the methods include automatically or at least semi-automatically extracting segmentations for training using a combination of heatmap based detection methods and text data processing. In particular, embodiments, the methods leverage expert text descriptions available in radiology reports. In comparison to the images, the radiology reports may be faster to annotate & train a prediction model for. It may be slow to review images due to the time to load in a viewer, browsing time to understand the contents and require expert analysis by radiologists to interpret. In addition, it may be particularly slow to create pixel-level annotations.

The following embodiments relate to independently extracting abnormal portions of medical image data and sets of entities and performing a matching process between the obtained extracted image portions and entities using clinically-informed feature sets to obtain training data. This may allow CAD systems to be efficiently built from large imaging datasets. In some embodiments, given a large dataset of medical images and associated text reports, containing examples of the pathologies that should be detected, the method includes performing an anomaly and/or abnormal portion detection process, on the medical image data and an entity detection process on the associated text reports to obtain a representation of anomalies and a representation of entities and performing a further matching process to match identified anomalies/abnormal portions with identified entities.

The identification of abnormal portions of a medical image is performed using one or more models. These models include artificial neural network or other machine learning based models. The identification process may comprise, for example, detecting the presence of abnormal medical image data and/or determining the presence and/or location of one or more anomalies, for example representing or associated with pathologies, in the medical image data. Such abnormal portions may include an anomalous sample that is an outlier compared to the normal distribution. For example, focal anomalies corresponding to anomalous regions associated or indicative of pathologies are of interest. These include, for example, tumour, haemorrhage, cyst, without limitation. Depending on the model used and the training of the model, other anomalies in the medical image data may be detected such as artefacts, for example, streak artefact, movement artefact and/or or implants for example, prosthetic eye, metal pins and/or abnormal anatomy.

In addition, the method includes a step of identifying entities in text data, for example, electronic health records, using one or more models. These models include artificial neural network or other machine learning based models. In general, the text data may correspond to or be obtained from any suitable free text or unstructured, or at least semi-structured document. In the following described embodiments, the text data corresponds to medical text data obtained from radiology reports that correspond to one or more medical images. The text data may be an output of a review of the medical images by a medical professional or other person, and include observations by the medical professional of the images. In this context, an entity may correspond to a finding and/or an impression of the medical professional and/or any observation of a real world object or anomaly in the image. A finding may be understood as an observation in the medical image and an impression may be understood as a diagnosis based on the findings. In some embodiment, an identified entity may comprise a pairing of both a finding and an impression resulting from the finding. Such entities can correspond to a disease, a symptom, a medication and/or other observable or observed property of the image and may be associated with a pathology. The entities may be findings and impressions reported by the radiologist.

In a medical context, the text to be analyzed may be a clinician's text note. The clinical text note may be stored within an Electronic Medical Record. The clinical text note may be a free-text radiology report. The text may be analyzed to obtain information about, for example, a medical condition or a type of treatment.

A data processing apparatus 10, also referred to as apparatus 10, according to an embodiment is illustrated schematically in FIG. 1. In the present embodiment, the data processing apparatus 10 is configured to process medical imaging data and to process medical text data. In other embodiments, the data processing apparatus 10 may be configured to process any other appropriate data, for example, any appropriate image or text data which may not be medical.

The data processing apparatus 10 comprises a computing apparatus 12, which in this case is a personal computer (PC) or workstation. The computing apparatus 12 comprises a processing apparatus 14. The computing apparatus 12 is connected to a display screen 16 or other display device, and an input device or devices 18, such as a computer keyboard and mouse.

The computing apparatus 12 is configured to obtain image data sets from a first data store, also referred to as a medical image data store 20. The image data sets may be generated by processing data acquired by a scanner 22 and stored in the data store 20.

The scanner 22 is configured to generate medical imaging data, which may comprise two-, three- or four-dimensional data in any imaging modality. For example, the scanner 22 may comprise a magnetic resonance (MR or MRI) scanner, CT (computed tomography) scanner, cone-beam CT scanner, X-ray scanner, ultrasound scanner, PET (positron emission tomography) scanner or SPECT (single photon emission computed tomography) scanner. The medical imaging data may comprise or be associated with additional conditioning data, which may for example comprise non-imaging data. The image data may be 1D, 2D, 3D or 4D data and/or wherein the medical image data comprises at least one of: CT, MRI, fluoroscopy, ultrasound data or medical imaging data obtaining using other modality; ECG data or other medical measurement data; volumetric data or slice data; and/or time series data.

The computing apparatus 12 may receive medical image data or other data from one or more further data stores (not shown) instead of or in addition to data store 20. For example, the computing apparatus 12 may receive medical image data from one or more remote data stores (not shown) which may form part of a Picture Archiving and Communication System (PACS) or other information system.

The computing apparatus 12 receives medical text data from a second data store, also referred to as medical data text store 24. In alternative embodiments, computing apparatus 12 receives medical text data from one or more further data stores (not shown) instead of or in addition to data store 24. For example, the computing apparatus 12 may receive medical text data from one or more remote data stores (not shown) which may form part of an Electronic Medical Records (EMR) or Electronic Health Records (EHR) system or Picture Archiving and Communication System (PACS). In some embodiments, the computing apparatus 12 receives medical image data and medical text data from a single data store.

Computing apparatus 12 provides a processing resource 14 or processing circuitry or automatically or semi-automatically processing medical image data and medical text data, for example, for the purpose of obtaining training data.

The processing apparatus 14 comprises image data processing circuitry 102 for obtaining identified abnormal regions of one or more images; text data processing circuitry 104 for obtaining one or more entities from text data; data matching circuitry 106 for performing a matching process using the identified abnormal regions and the identified entities to synthesize matched data; training circuitry 108 configured to train at least one further machine learning model using the matched data; and further image processing circuitry 110 configured to apply the trained model(s) to unseen image data.

In the present embodiment, the circuitries 102, 104, 106, 108 and 110 are each implemented in computing apparatus 12 by means of a computer program having computer-readable instructions that are executable to perform the method of the embodiment. However, in other embodiments, the various circuitries may be implemented as one or more ASICs (application specific integrated circuits) or FPGAs (field programmable gate arrays). In particular, the matching, training and further application of the trained models may be performed on different processing resources. In general, in the following described embodiments, the use of models is described and it will be understood that using a model may comprises applying a model to input data to generate output data. Using a model may comprise providing input data in a pre-determined data structure to a set of rules or an algorithm and outputting said output data. The model may be pre-determined through a separate training process. The pre-determined model may be a partially trained or a trained model.

The computing apparatus 12 also includes a hard drive and other components of a PC including RAM, ROM, a data bus, an operating system including various device drivers, and hardware devices including a graphics card. Such components are not shown in FIG. 1 for clarity.

The data processing apparatus 10 of FIG. 1 is configured to perform methods as illustrated and/or described in the following, for example, the method described with reference to FIGS. 2 and 3.

The apparatus 10 of FIG. 1 is configured to perform methods as described in the following. In other embodiments, different apparatus may be used to perform different processes, or parts of processes, of those described below. For example, a first apparatus may be used to perform the image analysis and abnormal portion detection, a second apparatus may be used to perform the entity identification, a third apparatus may be used to perform the matching process to generate further training data, a fourth apparatus may be used to train at least one further model using the generated training data, and a fifth apparatus may be used to apply the at least one further model to new image data. As such, any suitable combination of apparatuses may be used.

FIG. 2 depicts a method 200 of obtaining matched data. The method may be a method of obtaining data for training, refining and/or tuning a computer aided diagnosis algorithm. The method may include the step of assigning data and selecting the assigned data. The outputted matched data relates to annotated segmentation data that may be used to train further models.

At step 202, image data and associated text data is obtained. In the embodiment of FIG. 2, the image data comprises medical image data in the form of CT scan data. In the embodiment of FIG. 2, the text data corresponds to medical text data obtained from radiology reports that correspond to one or more medical images, however, the text data may be any suitable medical text data, as described above. In general, the medical text data comprises descriptive information, for example, medically and/or clinically relevant information and wherein the entity detection process comprises extracting said descriptive information and representing said information in the entity representation. The text data represents or is derived from radiology reports.

At step 204, an anomaly detection process is performed, for example by image data processing circuitry 102, on the image data using a first, trained model. The anomaly detection process is a pixel-wise anomaly detection process. The anomaly detection method results in a set of n_{,a,I} candidate anomalies extracted from I images. As described below with reference to FIG. 3, the candidate anomalies and/or abnormal regions may be extracted at different intensity threshold from anomaly heatmaps.

At step 206, an entity detection process is performed on the text data, for example by text data processing circuitry 104, using a second trained model. The text data corresponds to text report. The entity detection process results in a set of n_{e,i} candidate entities extracted from the corresponding i text reports. It will be understood that for each image and report pairing, the number of extracted abnormal regions does not correspond to the number of extracted entities. As described with reference to FIG. 4, the entity detection process provides entities and a set of corresponding attributes for each entity.

At step 208, a matching process is performed, for example, by data matching circuitry 106, between the first representation and the second representation. In the present embodiment, the matching process is modelled as a combinatorial optimisation assignment problem, in which the best fitted match between entities and anomalies is obtained. In the present embodiment, best fitted corresponds to a lowest cost of an entity-anomaly cost function. The matching process therefore includes the step of minimising such a cost function.

The entity-anomaly cost function can be constructed using quantities such as the following: features of the candidate anomaly ROI (intensity, texture, shape), the location of the image ROI (for example, images are registered to images to an atlas thus have a common coordinate system & mapping of anatomical regions), the absolute abnormality score of the image ROI, the attributes of the entity (anatomical location, severity, chronicity, size, certainty), the radiologist who wrote the report (for example, a level of detail and content of report may differ between radiologists). In some embodiments, a best match may be computed using any suitable assignment algorithm, for example, the Jonker-Volegenant algorithm may provide benefits. The matching process is described in further detail below.

In some embodiments, the cost function is based on further information determined from the medical text data. For example, author information or other measure of quality of the report may be used. Other metadata associated with the text may be used. Authors might be themselves considered categories, or the seniority of the author may be used as relevant information. For example, a junior radiologist, consultant radiologist, specialist consultant radiologist e.g. neuroradiologist, may be used. As a non-limiting example, these categories may be expressed as a one-hot vector. In some embodiments, the cost function has a term rewarding text representations derived from the same radiologist or author.

The following description of matching process are provided, however, it will be understood that alternative matching processes may be performed. In general, the matching process comprises obtaining a mapping between the obtained abnormal image portions and the entities based on one or more properties derived from the image and the attributes extracted from the text. The matching process includes a pair-wise (or, in some cases, a group-wise) evaluation of the matches between each of the detected abnormal portion and entities and assigning a score representative of a match between the anomaly representation and the entity representation. The score may be referred to as a matching score. Each pairing (or grouping) therefore has a resulting matching score. The process includes selecting one or more pairs (or groups) based on the score. In some embodiments, the selection is based on the highest matching score. In some embodiments, the matching is a combinatorial optimisation assignment problem and involves a group or pair wise assignment process to find a best match between identified anomalies and entities. The properties and attributes used in the matching process are clinically informed.

In the present embodiment, performing the matching process includes generating at least a first representation, for example, a feature vector, for each identified abnormal portions and at least a second representation, for example, a further feature vector, for each identified entity. The matching process includes evaluating the two representations and obtaining a score representing a degree of match between them. The matching of identified abnormal portions to entities is then based on the obtained score. In some embodiments, the representations may be combined when computing the cost function, for example, the feature vectors may be combined as a single representation such as a matrix or other multi-dimensional array.

The feature vectors are generated based on properties of the identified portions and corresponding attributes of the abnormal regions, for example, the feature vector for a first abnormal region incorporates properties of that abnormal region. The feature vector for an entity incorporates attributes of the entity. By constructing feature vectors in from image properties that correspond or are at least related to the attributes of the entities, a degree of match can be calculated using mathematical operations, such as a cosine similarity or other inner product based functions. In some embodiments, the properties selected for the image portion feature vector have a one-to-one correspondence to the properties selected for the entity feature vector. The matching process thus comprises obtaining a pair-wise (or group wise) degree of match between each identified region and each identified entity based on their corresponding properties and attributes, respectively, and selecting the closest matched pairs

At step 210, the matched data including paired or assigned matches are stored together as training data. The matched data provides training data in that the image portions assigned to an entity provide segmentation ground truth for a training process of a further model. The training data offers the abnormal regions/anomalies as segmentation ground truth for the class of matched entity.

At step 212, an evaluation step is performed on the matches. The evaluation step may include human input. Poor/failed matches can be either discarded or presented to human experts for review/annotation. The evaluation step may be a separate step for generating training data and/or verifying results or may form part of the training at step 24. An example evaluation step, is described with reference to FIG. 6.

At step 214, one or more further models are trained using at least the matched data, for example, by training circuitry 108 or by further model refining circuitry. In the present embodiment, a computer aided diagnosis model is trained. In some embodiments, the matched data is provided as input to a training data generation procedure for generating synthesised training data.

At step 216, an optional step of re-training and/or refining the anomaly detection algorithm is performed, for example, by training circuitry 108. In such a step, the matched data, or other output data, can be used to refine parameters of the model used at step 204 as part of a semi-supervised learning or training process.

FIG. 3 depicts data flow and data processing steps corresponding to method 200 in diagrammatic form. Image data 302 representing input medical images and text data 304 in the form of reports corresponding to the image input medical images are obtained, as described with reference to step 202. The image data 302 represents input medical images of a region of interest and the text data corresponds to medical text data regarding corresponding to the medical images of the region of interest. The image data 302 is provided as an input to an anomaly detection model 306, in this embodiment, an artificial neural network, to perform an entity detection process, as described with reference to step 204. The output of the anomaly detection process corresponds to identified abnormal portions of the medical image corresponding to potential anomalies. In this example, the abnormal portions correspond to a first abnormal portion 310a about the back periphery of the brain scan region, a second abnormal portion 310b on the left hemisphere of the brain and a third abnormal portion 310c on the left hand side of the brain region.

The text data 304 is provided as input to an independent entity detection model 308 to perform an entity detection process, as described with reference to step 206. In this embodiment, entity detection model 308 is an artificial neural network. The output of the entity detection model 308 is represented by a first identified entity 314a corresponding to “a hypodense area”, a second identified entity 314b correspond to an “infarct” and a third identified entity 316c correspond to a “sulcal effacement”. It will be understood that the hypodense area and infarct may be identified as a single entity, in some embodiments, and correspond to a finding and impression pairing.

The anomaly detection model 306 and the entity detection model 308 are independent and pre-determined through training on training data. The models 306 and 308 represent multi-layered artificial neural networks that are trained by optimising parameters of the neural network by minimising this difference between the predicted and measured quantities using suitable techniques such as minimising an error or cost function. It will be understood that other neural network or machine learning based optimization techniques may be used.

A matching process is then performed on the output of the two models, in accordance with embodiments. In particular, the matching process is performed between the one or more identified abnormal portions, identified by the anomaly detection process and the one or more identified entities identified by the entity identification process. As generally indicated by reference 316, the first abnormal region is matched to the second identified entity and the second abnormal region is matched to the first and/or second identified entity. The third abnormal region is not matched to any identified region. The matching process is performed as described with reference to step 208. In general, the matching process is performed based on properties of the identified abnormal portions and the attributes of the identified entities.

The result of the matching process is matched data represented by 318. The matched data represents a labelled or annotated image suitable for use in training one or more further models, for example, a computer aided diagnosis model. The matched data therefore represents groupings of one or more identified abnormal portions and one or more identified entities.

In some embodiments, the method comprises the step of training the further model(s). The output may be stored, as described with reference to step 210, or may be further evaluated, as described with reference to step 212 or may be used to train one or more further models, as described with reference to step 214.

The optional model refinement step (corresponding to step 216) is depicted by reference numeral 320 in which the anomaly detection algorithm is retrained and/or refined using a supervision from the matched anomaly pseudo-labels. This is in contrast to the initial unsupervised training of the model.

In the method of FIG. 2 and FIG. 3, an abnormal portion or anomaly detection image processing method is described in which a medical image is provided as an input to a pre-determined neural network or other suitable model and the model outputs a number of identified abnormal portions.

A non-limiting example of a model for detection of abnormal portion or anomaly detection, is a denoising autoencoder. For example, the autoencoder described in “Denoising Autoencoders for Unsupervised Anomaly Detection in Brain MRI”, by O'Neil et al, Proceedings of The 5th International Conference on Medical Imaging with Deep Learning, the content of which are hereby incorporated by reference. Such an example has a U-Net structure (as described at Section 4.3 and Appendix A) and is trained on a dataset of healthy brain MRI scans (4 sequences each: T1, T1Gd, T2, FLAIR).

The encoder-decoder architecture has three downsampling/upsampling stages. Each encoder stage consists of two weight-standardized convolutions with kernel sizes of 3 and 64, 128, 256 output channels for the three stages respectively followed by Swish activations and group normalization. Average 2×2 pooling is used for downsampling. The decoder architecture mirrors the encoder in reverse, using transposed convolutional layers for up-sampling.

In the above described method, application of the deep-learning model, for example, a denoising autoencoder and encoder model, a learned per-pixel anomaly score is output. In some models, a comparison between image and a normal distribution is performed. Each pixel/voxel of the heatmap has an anomaly score having a value that represents or indicates a probability that the pixel or voxel is anomalous.

the method is a heatmap based approach which involves generating pixel level heatmaps based on the difference between the image and the normal distribution.

As such, the application of the model allows for the identification of abnormal portions without any classification or labelling being performed solely based on the spatial distribution.

In some embodiments, applying the model comprises comparing the image data to an average or normal spatial distribution of the region of interest, as learned from training data of healthy subjects and identifying abnormal regions based on the comparison. In some embodiments, each pixel/voxel has a value representing the degree of difference over the learned normal distribution.

In some embodiments, the abnormal portions are identified by applying a number of different threshold levels. It will be understood that high threshold would capture only the most likely anomalous regions. For example, if a high threshold yielded two anomalous regions but three entities were extracted from the corresponding radiology report, the threshold for identifying the anomalous regions may be lowered to identify also weaker candidate anomalies.

The identifying of abnormal medical image data may comprises detecting the presence of abnormal medical/image data and/or determining the presence and/or location of one or more anomalies, for example representing or associated with pathologies, in the medical image data. As such, any suitable anomaly detection algorithm may be used. In particular, unsupervised heatmap based algorithms, such as those described in “Anomaly Detection via Context and Local Feature Matching”, 2022 IEEE 19th International Symposium on Biomedical Imaging (ISBI), IEEE, the contents of which are hereby incorporated by reference, may be suitable. Such methods can be extended to use human annotations where available to do semi-supervised learning.

FIG. 4 depicts an input medical image 402 corresponding to a CT scan of a brain. FIG. 4 depicts an intermediate heatmap of the image 404. FIG. 4 also depicts outputs of the abnormal region identity model in which 5 abnormal portions are identified. These include a first portion 414 at a first, high threshold, three portions 408, 410, 412 at a second threshold and a further portion 406 at a low threshold. In practice a high threshold is applied first and then apply lower thresholds to identify additional regions

Thresholding can be understood as selecting all pixels/voxels with an anomaly score over a selected threshold score as being positive detections. In addition, a morphology or connected components may be applied to the result. Morphology can be understood as performing spatial operations like dilation, erosion opening, closing in order to make detected regions more spatially coherent, for example, by removing random single pixel detections or single pixel holes in detected regions, smoothing “ragged” edges. In addition, connected components may be identified, in which regions of connected (i.e. neighbouring) pixels/voxels are identified and discard smaller regions which don't have a selected minimum number of components (pixels/voxels) connected.

In the present embodiments, an entity generally refers to a real-world object, such as a finding and/or an impression. Entities may correspond to pathologies and may related to effects of a disease, or symptom of a diseased or effect of a medication. The text extraction described above is performed using any suitable text extraction algorithm. For example, the text extraction algorithm found in “Templatedtext synthesis for expert-guided multi-label extraction from radiology reports”. Schrempf et al., 2021 Machine Learning and Knowledge Extraction, 3(2), pp. 299-317. That method uses a neural network Transformer model PubMedBERT as a base model. FIG. 5 is a schematic diagram of a set of 33 labels for entities that relate to neurological abnormalities from head CT reports for stroke patients. The model was trained on a dataset contains 28,687 radiology reports supplied by the West of Scotland Safe Haven within NHS Greater Glasgow and Clyde (GGC). FIG. 5 depicts 13 radiographic findings (labelled 502), 16 clinical impressions (labelled 504) and 4 crossover labels which are indicated with a single asterisk. Finding to impression links are shown schematically in FIG. 5. A number of labels fit both the finding and impression categories and are labelled by an asterisk. Example pathologies associated with findings and impressions are also labelled 506.

In addition to extraction of entity extraction from the text data, the method also includes extraction of attributes of entities from the text. In particular, in addition to the deep learning model for identifying entities, a further deep learning model is provided to obtain entity attributes associated with the identified entities. Such attributes may relate to, for example, the location and appearance of the entity. In some embodiments, obtaining the entities and their attributes is a two stage process, in which the text data is provided as input to a first trained model to extract the entities. The entities are then provided as input to a second model, together with the text and the output from the model is a set of values for attributes.

As a non-limiting example, in one embodiment, two BERT (Bidirectional Encoder Representations from Transformers) models are chained together. BERT models are described in “BERT: Pre-training of Deep Bidirectional Transformers for Language Understanding”, by Devlin et al.

The model allows, for each pathology, the following attributes to be identified and annotated in the text data: Attributes may be related to polarity and/or certainty, for example, one of {Positive, Negative, Uncertain}. Attributes relating to laterality, for example, one of {Left, Right, Bilateral}. Attributes relating to Chronicity, for example, one of {Acute, Subacute, Chronic, Acute-on-Chronic}. Attributes relating to severity, for example, one of {Mild, Moderate, Severe} or a change in severity, for example, one of {Unchanged, Lessened, Worsened}. Attributes relating to Anatomical Location, for example, Tissue (one of {White matter, Grey matter, Territory, Bone, Vessel, Dura}) or Region (lesions) (one of {extracranial, intracranial [extra-axial [ . . . ], intra-axial [ . . . ]}) or Artery (vascular pathology) (one of {anterior cerebral, middle cerebral [M1, M2, M3, M4], . . . }). The attribute may relate to Anatomical distribution (for example, one of {Focal, Global}). The above list of attributes is provided as a non-limiting example. At the point of annotation, the labels are represented as text as a human-readable name.

In one example, the entity identification model, is trained to obtain predictions for a certain set of labels for each sentence of the radiology report and/or for the radiology report as a whole. Each label relates to a corresponding entity, in this embodiment, a finding and/or an impression. For example, such labels may include haemorrhage and tumour. The deep learning model may be trained to classify each sentence or report as whole to say whether each of haemorrhage and tumour is present. As an example, for each sentence, each of the labels is classified in one of a plurality of certainty classes. The certainty classes include positive, uncertain and negative. A classification in the positive certainty class is made when the model determines from the sentence that the finding or impression that is represented by the label is present. A classification in the negative certainty class is made when the model determines from the sentence that the finding or impression that is represented by the label is not present. A classification in the uncertain certainty class is made when the model determines from the sentence that the presence of the finding or impression is uncertain. For example, the sentence may suggest that the finding or impression may be present without providing a strong enough indication to be classified as positive.

In the method 200 described above, a matching process was described. Further examples of the matching process are provided in the following. In further detail, for each possible injective matching function m: a_i→e_i, matching anomalies ‘a’ to entities ‘e’ described in the report for each image ‘i’, we can compute a cost function that we define. The cost function may include a number of terms. In the following, terms relating to or representing a goodness or closeness of the entity-anomaly match and intra-anomaly consistency of the anomaly groups are considered. In some embodiments, the cost function is representative or proportional to a degree of match between the properties of the abnormal portion and the attributes of the entities. The cost function may also be referred to as a matching function.

A first example of a term for a suitable cost function is an entity-anomaly matching cost function that uses cosine similarity to assess if the matched entity and anomaly have similar properties. The matching process thus comprises calculating a value of a cost function for each pairing of abnormal portions and identified entities and determining a pair having a lowest value of the cost function and/or one or more pairs having a value below a pre-determined threshold. The cost function is defined as follows:

$$C ( m ) = - {\sum }_{i = 0}^{{n}_{i}} {\sum }_{α = 0}^{{n}_{{α}_{i}}} {S}_{C} ( {f}_{{a}_{i}} , {f}_{m ( {a}_{i} )} )$$

The above cost function considers a potential match across all pairs. During the matching process, it is the combination of pairs with lowest total cost that we are interested in identifying. A score for each pairing of entity and anomaly is calculated and then summed together. The above cost function therefore outputs values representing a similarity between regions and entities.

The feature vectors fₐ and fₘ correspond to a first feature vector extracted from an image and a second feature vector extracted from associated text. As an example, the image feature vector represents properties of the anomaly, for example, properties derived from one or more image processing steps on the abnormal portion. In this example, the anomaly feature vector include entries corresponds to measured values of an anomaly region of interest (ROI). The entity feature vector corresponds to the attributes detected in the text description. These may be mapped to numerical values.

As a non-limiting example, a feature vector fₐ for an image portion (the anomaly feature vector) has the following entries obtained by measuring attributes of the anomaly region of interest: (a mean intensity of anomaly ROI; X co-ordinate of anomaly ROI centre; Size of anomaly (voxels)). The related feature vector fₘ₍ₐ₎ for each entity obtained by detecting attributes in the text description and mapping to numerical values, has the following entries: {“hypodensity”=0, “hypodensity=1}, {“left”=0, “bilateral”=0.5, “right”=1}, {“small”=0, “moderate”=0.5, “large”=1}. In some embodiments, the feature vectors are constructed such that there is a one to one correspondence between the at least one properties derived from the abnormal portion and the at least one attribute of the identified entity used in the matching process. In some embodiments, the relationship between the entries of the anomaly feature vector and the entity feature vector is pre-determined. In some embodiments, that relationship is learned through a further machine learning process.

The cost function might also include an additional term configured to penalise, during a training process, a high variance within a candidate entity group (as low variance means good consistency) to ensure that anomalies matched to the same entity type have consistent properties. For example, radiomics style features extracted from an ROI might be considered, such as, intensity mean, intensity variance, Size, Gray-level co-occurrence matrix (GLCM), fractal features. These features may be the same or different to the matching features described in the previous slide. Where m⁻¹ denotes the inverse of the matching function described in the previous slide (i.e. m⁻¹: a_i→e_i) and we have a set of n_{f} features f, a term for variance may be added. In further detail, in such an example, the cost function is:

$$C ( m ) = - {\sum }_{i = 0}^{{n}_{i}} {\sum }_{α = 0}^{{n}_{{α}_{i}}} {S}_{C} ( {f}_{{a}_{i}} , {f}_{m ( {a}_{i} )} ) + {\sum }_{i = 0}^{{n}_{f}} {\sum }_{ϵ = 0}^{{n}_{e}} Var ( f ( {m}^{- 1} ( {ϵ}_{i} ) )$$

The features f for which variance is penalised could be different for each entity e. In some embodiments, a feature reduction process is performed to obtain a reduced/compressed feature set, for example, using principle component analysis and one or more parts of the cost function are applied to the reduced/compressed feature set. For example, the measure of variance may be applied to the reduced features rather than the original features. In the above cost function, the total number of features is n_f, the total number of entities is n_e. The second term iterates through all features and all entities, and sum across the variance for all pairs. The variance violates independence and requires consideration of the set (combination) of pairs. Therefore the second term does not loop over entities & anomalies but over features & entities (and then computes for all anomalies assigned to a given entity label, as part of the matching). The above cost function therefore has a first term that outputs values representing a similarity between regions and entities and a second term that penalizes variation of abnormal image portions assigned to the same class of entities.

In embodiments, in which some ground truth is available, for example, in circumstances in which a known set of anomaly-entity matches is available, then rather than measuring simple variance, as described above, a distance of feature values for assigned anomalies from the feature values for the known true anomalies may be used.

The method may comprise applying an optimisation process to find the lowest cost (and therefore best) mapping between entities to anomalies. The optimisation process may be based on, for example, the Jonker-Volegenant algorithm applied to solve as multiple linear assignment problems.

FIG. 6 depicts an example evaluation step in which user input is requested. FIG. 6 is presented as an illustrative example and depicts two proposed matches based on the data described with reference to FIG. 3. A graphical representation 602 of the two proposed matches are presented to a user, for example, on display screen 16, together with an interactive element for confirming or rejecting the matches determined. In this example the interactive elements are tick boxes. The example user interface allows a user to quickly verify matched data, for example, via input device 18. In some embodiments, the displayed matched data includes groupings of identified abnormal portion(s) and entities and then receiving further user input representing the evaluation by the user. The user input may represent confirmation of the match or a rejection of the match. The evaluation step may form part of a training method. The method offers the opportunity for radiologists to view and edit the matches (as well as the matching features), between image regions and lines of text. This allows controllability by domain experts at the voxel and/or region level, without the requirement of a full-scale voxelwise annotation.

Although the embodiments above are described with regard to medical data, in other embodiments any text and/or image data may be processed using methods described above.

In the above described embodiments, the entity detection models are unsupervised anomaly detection where the idea is to learn what the normal distribution looks like and then detect “outliers” from the distribution at test time. However, in further embodiments, the method may also be performed using anomaly detection methods trained on datasets including both normal & abnormal images and/or potentially trained on an unlabelled dataset of all images where the method learns to discover the outliers within the training data distribution. It is assumed that there are not sufficient labelled anomalies to train a fully supervised algorithm that already detects specific anomalies (for example, labelled as haemorrhage, tumour, etc.) and therefore we want to leverage information from the associated radiology reports in order to assign (match) these labels.

Certain embodiments provide a method for detecting radiological findings. The method may comprise a semi-supervised anomaly detection method for medical imaging data, that can be trained using a dataset of healthy images and abnormal images, to yield heatmaps showing the degree of anomaly at each pixel. The method may comprise extracting regions of interest from the anomaly heatmap. The method may comprise an entity recognition method for corresponding text radiology reports. The method may comprise a method for extracting attributes (e.g. laterality, size, severity) of each entity in the text radiology reports. The method may comprise creating representations of the imaging anomalies. The method may comprise creating representations of the text entities. The method may comprise a matching function to quantify the quality of the match between each anomaly/entity representation pair i.e. the “cost function”. The cost function may be referred to as a matching function. The method may comprise an optimiser to find the lowest cost (best) mapping from entities to anomalies. The optimiser may be based on, for example, the Jonker-Volegenant algorithm applied to solve as multiple linear assignment problems.

The cost function may be a cosine similarity between image and text vector representations, with the representations constructed such that equivalent elements represent equivalent properties.

A cost term in the matching function may penalise high variation of the image representations for all anomalies assigned to the same entity class.

A cost term in the matching function may penalises high variation of the image representations for all anomalies with a similar corresponding text representation.

A cost term in the matching function that rewards lower variation of the image representations for all anomalies with a similar corresponding text representation, for text representations derived from the same radiologist rather than a different radiologist.

The image representation may be learned using a deep learning method. The abnormal portion of the image and/or the entities are obtained using deep learning model. The text representation may be learned using a deep learning method. Other suitable image analysis and natural language processing methods may be used to obtain the abnormal portion of the images and text.

The method may comprise retraining the anomaly detection method using also supervision from the matched anomalies. The method may comprise training on both healthy data and matched pathological data.

The method may comprise an evaluation step, for example, in which a human user reviews the automatic matches and filters/selects correct matches, before using these human-confirmed matches as additional supervision.

The method may comprise retraining the anomaly detection method using also supervision from the matched anomalies. The method may comprise training on both healthy data and matched pathological data.

The method may comprise an evaluation step, for example, in which a human user reviews the automatic matches and filters/selects correct matches, before using these human-confirmed matches as additional supervision. The evaluation step may comprise displaying a user interface to a user via a display that user interface depicting matched pairs or other output.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments are described, these embodiments have been presented by way of example only, and are not intended to limit the scope of the invention. Indeed the novel methods and systems described herein may be embodied in a variety of other forms. Furthermore, various omissions, substitutions and changes in the form of the methods and systems described herein may be made without departing from the spirit of the invention. The accompanying claims and their equivalents are intended to cover such forms and modifications as would fall within the scope of the invention.

1. An apparatus, comprising:
processing circuitry configured to:
obtain image data representing one or more medical images of a region of interest and processing said image data to identify one or more abnormal portions of the image by applying a first pre-determined model to the obtained image data;
obtain medical text data corresponding to the one or more medical images of the region of interest and process said medical text data to identify one or more entities and their associated attributes by applying at least one further pre-determined model to the obtained medical text data; and
perform a matching process between the one or more identified abnormal portions and the one or more identified entities to obtain matched data comprising groupings of at least one identified abnormal portions and at least one entity,
wherein the matching process is based on at least one or more properties of the identified abnormal portions of the image data and at least one or more of the attributes associated with the identified entities.

2. The apparatus of claim 1, wherein the matching process comprises generating at least one representation of the identified abnormal portions and at least one further representation of the identified entities and obtaining a score representative of a degree of match between the first and second representations and matching the abnormal portions to at least one of the identified entities based on the obtained score.

3. The apparatus of claim 1, wherein the processing circuitry is further configured to store the matched data and/or use the matched data to train at least one further model and/or generate training data for at least one model using said matched data.

4. The apparatus of claim 1, wherein identifying the one or more abnormal portions comprises identifying portions in comparison to a pre-determined or learned distribution for healthy and/or normal data based on a difference between a spatial distribution of the medical image and a pre-determined normal distribution, for example, at a pixel or voxel level and/or as represented by a heatmap.

5. The apparatus of claim 1, wherein the identification of the one or more abnormal regions used a pixel or voxel level approach, for example, thresholding, morphology or connected components based approach.

6. The apparatus of claim 1, wherein obtaining the entity and their attributes comprises applying at least one first model to the text data to identify said entities and applying at least a second model to obtain the attributes associated with the entities.

7. The apparatus of claim 1, wherein the matching process comprises determining a degree of match based on similarity and/or a consistency between properties of the one or more abnormal portions and attributes of the one or more entities.

8. The apparatus of claim 1, wherein the matching process comprises determining a similarity function or other measure of distance between mathematical representations of the one or more properties of the abnormal image portions and the attributes of the identified entities and their attributes.

9. The apparatus of claim 1, wherein the matching process is based on a pre-determined relationships between the one or more properties and the one or more attributes.

10. The apparatus of claim 1, wherein the matching process is based on minimizing or otherwise optimizing a matching function, the matching function comprising a term representing similarity between the identified abnormal regions and/or a term penalizing a variation of abnormal image portions assigned to the same class of entities.

11. The apparatus of claim 1, wherein the matching process comprises performing an optimisation optimization process, for example, the Jonker-Volegenant algorithm applied to solve as multiple linear assignment problems.

12. The apparatus of claim 1, wherein the processing resource is further configured to retrain and/or refine the first and/or the second model using the obtained matched data.

13. The apparatus of claim 1, wherein the processing resource is further configured to display the matched data, for example, the groupings of one or more abnormal portions and entities to a user and obtaining further user input representing a user evaluation of the matched data as part of a further training process or as part of generation of training data.

14. The apparatus of claim 1, wherein the first and/or the second model comprises a deep learning or other artificial neural network based model.

15. The apparatus of claim 1, applying a principle component analysis or other feature reduction procedure to a larger set of features, and wherein at least part of the matching process is applied to the reduced set of features

16. The apparatus of claim 1, wherein the medical image data comprises 1D, 2D, 3D, or 4D data, and/or wherein the medical image data comprises at least one of:
CT, MRI, fluoroscopy, ultrasound data, or medical imaging data obtaining using other modality;
ECG data or other medical measurement data;
volumetric data or slice data; or
time series data.

17. The apparatus of claim 1, wherein the one or more properties of the identified abnormal region comprise at least one of: intensity, texture, shape, location, and a measure of abnormality of at least the abnormal portion.

18. The apparatus of claim 1, wherein the one or more entities comprise at least one of a finding, an impression or other observable, wherein the entity is associated with a pathology, and wherein the one or more attributes associated with the entity may comprise attributes associated with anatomical location or region, an anatomical distribution, laterality, severity, or a level of certainty.

19. The apparatus of claim 1, wherein the matching process is further based on further information obtained from the medical text data, for example, author information and/or a measure of quality and/or content of the medical text data and/or other metadata.

20. A method, comprising:
obtaining image data representing one or more medical images of a region of interest and processing said image data to identify one or more abnormal portions of the image by applying a first pre-determined model to the obtained image data;
obtaining medical text data corresponding to the one or more medical images of the region of interest and process said medical text data to identify one or more entities and their associated attributes by applying at least one further pre-determined model to the obtained medical text data; and
performing a matching process between the one or more identified abnormal portions and the one or more identified entities to obtain matched data comprising groupings of at least one identified abnormal portions and at least one entity,
wherein the matching process is based on at least one or more properties of the identified abnormal portions of the image data and at least one or more of the attributes associated with the identified entities.
